(19) United States
(12) Patent Application Publication
(10) Pub. No.: US 2019/0119496 A1
(43) Pub. Date: Apr. 25, 2019

(54) Totally bio-based vegetable oil polyol and preparation method and use thereof

(71) Applicant: NANJING TECH UNIVERSITY, Nanjing (CN)

(72) Inventors: Kai GUO, Nanjing (CN); Zheng FANG, Nanjing (CN); Chengkou LIU, Nanjing (CN); Ning ZHU, Nanjing (CN); Jingjing MENG, Nanjing (CN); Junjie TAO, Nanjing (CN); Xin HU, Nanjing (CN); Xin LI, Nanjing (CN); Chuanhong QIU, Nanjing (CN); Pingkai OUYANG, Nanjing (CN)

(21) Appl. No.: 16/221,354

(22) Filed: Dec. 14, 2018

(30) Foreign Application Priority Data
Sep. 29, 2018 (CN) 201811153270.0

(51) Int. Cl. C08L 91/00; C07D 303/42; B01J 19/00

(52) U.S. Cl. CPC C08L 91/00; C08G 18/627; B01J 19/0093; C07D 303/42

(57) ABSTRACT

A method comprises enabling epoxy vegetable oil to react with a compound of a formula III in a second microstructured reactor to obtain the vegetable oil polyol. Compared with the existing technology, the present invention adopts a novel, environment-friendly ring-opening agent, the obtained polyol is novel in structure, high in hydroxyl value, even in distribution and low in viscosity, and can completely replace traditional petrochemical polyol to be applied to the preparation of polyurethane foam materials.

This application claims priority to Chinese Patent Application Ser. No. CN201811153270.0 filed on 29 Sep. 2018.

### TECHNICAL FIELD

The present invention relates to the field of chemical materials and production techniques thereof, particularly to a totally bio-based vegetable oil polyol and a preparation method and use thereof. The totally bio-based vegetable oil polyol synthesized by the present invention is suitable for preparing polyurethane materials.

### BACKGROUND ART

Polyurethane is prepared by reaction of isocyanate and polyol, and has a carbamate chain segment repeating unit structure, and has been widely applied to technical fields such as foamed plastics, surface coatings, adhesives, encapsulants and complexing agent materials. Polyurethane materials have excellent performance, wide application and diversified products, wherein polyurethane foaming plastic has the widest application. Recently, researchers around the world are forced to prepare polyurethane with vegetable oil by the consumption of petrochemical fuel resources and the increasing concern for environmental issues.

Vegetable oil polyols are mainly used in the field of polyurethane preparation, and the prepared vegetable oil-based polyurethane materials are totally environmental friendly. Because of the hydrophobicity of fatty acid glycerides, the major components of vegetable oil, the vegetable oil-based polyurethane materials have superior physicochemical performance, especially better hydrophobicity and thermal stability. Thereby, the vegetable oil polyols and the polyurethane materials thereof develop quickly.

Vegetable oil polyols are important renewable resources, can react with isocyanate compounds to generate polyurethanes, and are good alternative raw materials for petroleum-based polyols. In recent years, main methods for synthesis of a vegetable oil polyol include: 1) performing alcoholysis reaction on vegetable oil and a polyol to generate a polyhydroxy compound; 2) generating a polyhydroxy compound with terminal hydroxyls by oxidizing unsaturated double bonds in the vegetable oil with ozone; 3) oxidizing the vegetable oil into epoxy vegetable oil, then processing through hydrolysis, hydrogenation, methyl esterification or halogenation to generate a polyhydroxy compound.

Among above methods for synthesis of vegetable oil polyols, 1) and 3) have wider use. CN1837180A and CN101139252A relate to methods for preparing vegetable oil polyol by a three-step reaction of alcoholysis, epoxidation and ring opening with rapeseed oil and Jatropha seed oil as the main raw materials respectively. CN10106016A relates to a method for preparing vegetable oil polyol by a two-step reaction of epoxidation and ring opening with rubber seed oil as the main raw material. CN1907944A relates to a method for preparing vegetable oil polyol by a two-step reaction of ring opening and alcoholysis with epoxy rapeseed oil as the main raw material. CN101659627A relates to a method for preparing vegetable oil polyol by simultaneously performing epoxy ring opening and ester group amidation reactions on epoxy vegetable oil and glycolamine.

The methods for preparing vegetable oil polyols provided by the above patents mainly based on epoxy ring opening, all react in batch reactors, and mainly have the following drawbacks: 1) long reaction time; 2) high energy consumption; 3) low equipment self-controlling level; and 4) low hydroxyl values and high viscosities of products due to the side reactions of crosslinking.

### SUMMARY OF THE INVENTION

The present invention aims to provide a totally bio-based vegetable oil polyol for the above-mentioned problems in the existing technology, and the totally bio-based vegetable oil polyol is novel in structure, high in hydroxyl value and low in viscosity, and can completely replace petrochemical polyol to be applied to the field of polyurethane foam materials.

Another objective of the present invention is to provide a preparation method of the totally bio-based vegetable oil polyol. The preparation method is environment-friendly and easy to operate, and the product does not need further processing.

A final objective of the present invention is to provide the use of the totally bio-based vegetable oil polyol in the preparation of polyurethane.

For realizing the above objectives, the present invention provides the technology solutions as follows:

The present invention provides a preparation method of a totally bio-based vegetable oil polyol, comprising enabling epoxy vegetable oil to react with a compound of a formula III in a second microstructured reactor to obtain the vegetable oil polyol

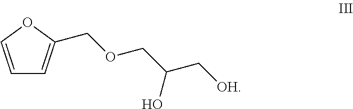 [SMILES: OCC(O)COCc1ccco1]

Preferably, the preparation method of the totally bio-based vegetable oil polyol comprises the following steps:

(1) simultaneously pumping a mixed solution of hydrogen peroxide, an organic acid, a catalyst and a stabilizer as well as the vegetable oil into a first microstructured reactor of a micro-channel modular reaction device for reacting to obtain a reaction solution containing the epoxy vegetable oil;

(2) simultaneously pumping the reaction solution containing the epoxy vegetable oil obtained from the step (1) and the compound of the formula III into the second microstructured reactor of the micro-channel modular reaction device for reacting to obtain the vegetable oil polyol

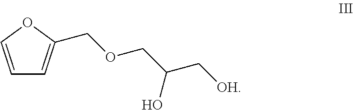 [SMILES: OCC(O)COCc1ccco1]

Most preferably, the preparation method of the totally bio-based vegetable oil polyol comprises the following steps:

(1) simultaneously pumping the mixed solution of the hydrogen peroxide, the organic acid, the catalyst and the stabilizer as well as the vegetable oil into a first micro-mixer of the micro-channel modular reaction device, uniformly mixing, then enabling a resulted mixed solution to flow into the first microstructured reactor of the micro-channel modular reaction device for reacting to obtain the reaction solution containing the epoxy vegetable oil;

(2) simultaneously pumping the reaction solution containing the epoxy vegetable oil obtained from the step (1) and the compound of the formula III into a second micro-mixer of the micro-channel modular reaction device, uniformly mixing, then enabling a resulted mixed solution to flow into the second microstructured reactor of the micro-channel modular reaction device for reacting to obtain the vegetable oil polyol

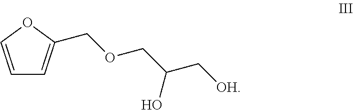 [SMILES: OCC(O)COCc1ccco1]

In the step (1), the hydrogen peroxide has a concentration of 25-35 wt %, preferably 30 wt %. The organic acid is formic acid or acetic acid. The catalyst is sulfuric acid or phosphoric acid, preferably sulfuric acid.

The stabilizer is ethylenediamine tetraacetic acid (EDTA). The vegetable oil is at least one selected from olive oil, peanut oil, rapeseed oil, cottonseed oil, soybean oil, palm oil, sesame oil, sunflower oil, linseed oil, tung oil, safflower oil, rice bran oil, corn oil and teaseed oil, preferably soybean oil or rapeseed oil, more preferably soybean oil. The mole ratio of the double bonds in the vegetable oil to the hydrogen peroxide to the organic acid to the catalyst to the stabilizer is 1:(6-20):(6-20):(0.02-0.4):(0.006-0.2), preferably 1:(12-20):(12-20):(0.2-0.4):(0.015-0.1).

In the step (1), the first microstructured reactor has a reaction temperature of 60-130° C., preferably 90° C. The reaction residence time is 5-10 min, preferably 8 min. The reaction pressure is normal pressure. The first microstructured reactor has a volume of 20-60 mL. The vegetable oil is pumped into the micro-channel modular reaction device at a flow rate of 0.5-1.0 mL/min, preferably 0.8 ml/min. The mixed solution is pumped into the micro-channel modular reaction device at a flow rate of 3.5-5.0 mL/min, preferably 4.7 ml/min.

In the step (2), the mole ratio of the epoxy groups in the epoxy vegetable oil to the compound of the formula III is 1:(1.5-4.5), preferably 1:(1.5-2.2).

In the step (2), the second microstructured reactor has a reaction temperature of 70-100° C., preferably 85° C. The reaction residence time is 6-10 min, preferably 8 min. The second microstructured reactor has a volume of 96-240 mL. The compound of the formula III is pumped into the second micro-mixer at a flow rate of 12.0-18.0 ml/min, preferably 16.5 mL/min.

In the step (2), the reaction effluent of the second microstructured reactor is introduced into an oil-water separator, wherein an aqueous phase is removed and an oil phase product is collected, thus obtaining the vegetable oil polyol.

In the step (2), the compound of the formula III is prepared by the following process, comprising:

(a) dissolving furfuryl alcohol (a compound of a formula I) in a reaction solvent, dropwise adding thionyl chloride at −10° C. to 10° C., continuing stirring and reacting for 0.5-2 h, adding water to quench the reaction, collecting an organic phase, and spin drying the reaction solvent to obtain colorless liquid (2-chloromethyl furan, a compound of a formula II);

(b) then adding glycerol and sodium into the colorless liquid, continuing stirring and reacting for 3-6 h at 30-50° C., to obtain the compound of the formula III.

A synthesis route of the compound of the formula III is as follow:

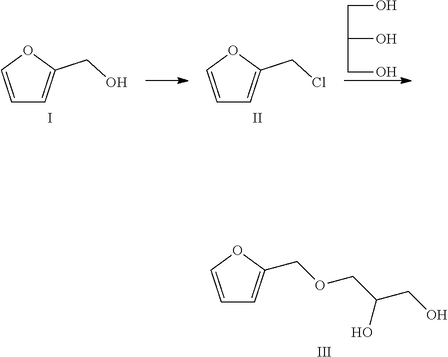

In the step (a), the reaction solvent is one or more of dichloromethane, dichloroethane, chloroform and benzene, preferably dichloromethane. The mole ratio of furfuryl alcohol to thionyl chloride to glycerol to sodium is 1:(1.0-2.0):(1.0-2.0):(1.0-2.0), preferably 1:(1.0-1.5):(1.0-1.5):(1.0-1.5).

Preferably, the compound of the formula III is prepared by the following process, comprising:

(a) dissolving the furfuryl alcohol (the compound of the formula I) in the reaction solvent, dropwise adding the thionyl chloride at −5° C. to 0° C., continuing stirring and reacting for 1-2 h and adding water to quench the reaction, collecting the organic phase, and spin drying the reaction solvent to obtain the colorless liquid (2-chloromethyl furan, the compound of the formula II);

(b) then adding glycerol and sodium into the colorless liquid, continuing stirring and reacting for 4 h at 35-40° C., to obtain the compound of the formula III.

The micro-channel modular reaction device comprises the first micro-mixer, a first microstructured heat exchanger, a first tubular temperature control module, the first microstructured reactor, the second micro-mixer, a second microstructured heat exchanger, a second tubular temperature control module and the second microstructured reactor which are sequentially connected through pipelines. The reaction materials are fed into the micro-mixer and subsequent equipment through precise pumps with low pulsation level.

Preferably, the micro-channel modular reaction device further includes an oil-water separator and a receiver. The discharging outlet of the second microstructured reactor, the oil-water separator and the receiver are sequentially connected.

The types of the first micro-mixer and the second micro-mixer are both slit plate mixer LH25.

The types of the first microstructured heat exchanger and the second microstructured heat exchanger are both coaxial heat exchanger.

The first tubular temperature control module and the second tubular temperature control module are used for precisely controlling the temperatures.

The types of the first microstructured reactor and the second microstructured reactor are meander reactor HC, sandwich reactor HC, fixed bed meander reactor HC or Hastelloy micro-channel reactor, respectively.

The totally bio-based vegetable oil polyol prepared by the method of the present invention.

The use of the totally bio-based vegetable oil polyol of the present invention in the preparation of polyurethane foam.

The vegetable oil contains unsaturated carbon-carbon double bonds, which generate epoxy groups by Prileshajev epoxidation. Then hydroxyl groups are introduced into the epoxy groups by ring opening reaction. Commonly used ring-opening agents include micromolecular alcohol, alcohol amine or carboxylic acid. As for a monofunctional ring-opening agent, the hydroxyl value of a product is low, and for a polyfunctional ring-opening agent, the viscosity of a product is high viscosity and the hydroxyl value is low due to the fact that hydroxyls are adjacent to each other, a monomeric ring-opening agent performs ring-opening reaction on epoxy groups in multiple grease molecules, and newly formed hydroxyls also participate the ring opening reaction, causing the grease molecule to be polymerized. The reaction between the furfuryl alcohol and the glycerol may introduce a furan ring into the ring-opening agent and retain only one primary hydroxyl, efficiently improving the mechanical properties of the product and reducing the viscosity of the product.

The present invention employs a special polyhydroxy compound as the ring-opening agent. The ring-opening agent is a totally bio-based polyhydroxy compound prepared with furfuryl alcohol and glycerol as starting materials. The polyhydroxy compound used in the present invention have a novel structure and a proper functionality, ensuring the vegetable oil polyol prepared by ring opening reaction of the polyhydroxy compound having lower viscosity and higher hydroxyl value, and the polyurethane foam material based on the vegetable oil polyol having excellent performance. Furthermore, the catalyst selected in the present invention is used in a very small amount such that the use of the polyol will not be impacted by a trace of the remaining catalyst and the product does not need further refinement, and the process is simple.

Beneficial effect: Compared with the existing technology, the present invention adopts a novel, environment-friendly ring-opening agent, the obtained vegetable oil polyol is novel in structure, high in hydroxyl value, even in distribution and low in viscosity, and can completely replace traditional petrochemical polyol to be applied to the preparation of polyurethane foam materials. Meanwhile, the preparation method of the present invention can realize continuous operation, the preparation process is simple and easy to control, the reaction time is short, the operation is convenient, the energy consumption is low, the side reaction is less, the reaction efficiency is high, the obtained product does not need further processing and is suitable for industrial production. In the aspect of reactive mode, the present invention adopts a micro-channel modular reaction device, which can efficiently increase the reaction efficiency, inhibit the occurrence of side reactions and reduce the energy consumption.

### BRIEF DESCRIPTION OF THE DRAWINGS

FIG. 1 shows a schematic diagram of a micro-channel modular reaction device. VO=Vegetable oil; HOCS=Hydrogen peroxide Organic acid Catalyst Stabilizer; MM=micro-mixer; MHE=microstructured heat exchanger; MR=microstructured reactor; TTCM=tubular temperature control module; AL=Aqueous Layer; OWS=oil-water separator.

### DETAILED DESCRIPTION OF THE INVENTION

The present invention will be better understood according to the following Examples.

The vegetable oil polyol and the polyurethane foam material prepared according to the present invention are analyzed with following methods:

(1) The hydroxyl value is measured according to GB/T 12008.3-2009;

(2) The viscosity is measured according to GB/T 12008.7-2010;

(3) The apparent density of foam plastics is measured according to GB/T 6343-2009;

(4) The compressive strength of rigid foam plastic is measured according to GB/T 8813-2008 with the cross section in the direction perpendicular to the foaming as the compression face, the compression rate of 5 mm/min and the measurement value at 10% deformation of a sample as the compressive strength of the material;

(5) The impact strength of rigid foam plastic is measured according to GB/T 11548-1989. The impact strength is used for characterizing the toughness under high speed impact or the resistance to fracture of the materials;

(6) The dimensional stability of rigid foam plastic is measured according to GB/T 8811-2008.

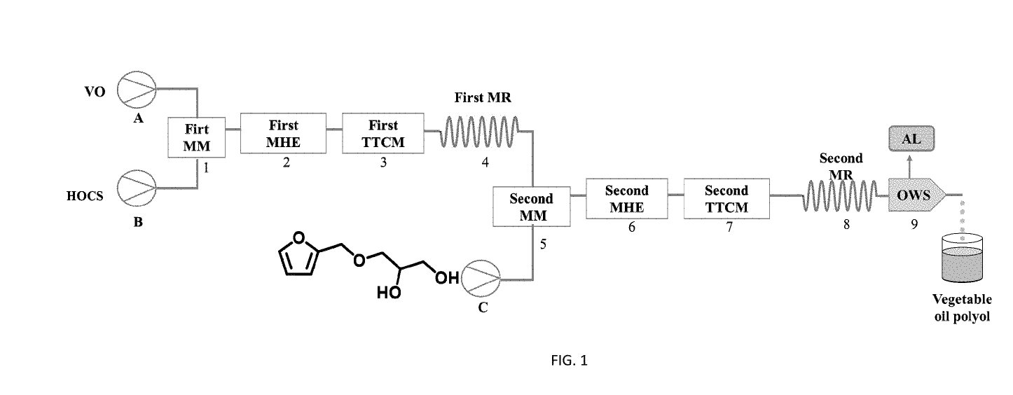

As shown in FIG. 1, a micro-channel modular reaction device described in the following examples includes a first micro-mixer, a first microstructured heat exchanger, a first tubular temperature control module, a first microstructured reactor, a second micro-mixer, a second microstructured heat exchanger, a second tubular temperature control module, a second microstructured reactor, an oil-water separator and a receiver which are sequentially connected through pipelines. The feeding inlet of the first micro-mixer is connected with a first liquid storage tank (a vegetable oil storage tank) through a pump A. The feeding inlet of the first micro-mixer is connected with a second liquid storage tank (a storage tank for a mixed solution of hydrogen peroxide, organic acid, catalyst and stabilizer) through a pump B. The feeding inlet of the second micro-mixer is connected with the discharging outlet of the first micro-reactor. The feeding inlet of the second micro-mixer is connected with a third liquid storage tank (a storage tank of a compound of a formula III) through a pump C.

The types of the first micro-mixer and the second micro-mixer are both plate mixer LH25.

The types of the first microstructured heat exchanger and the second microstructured heat exchanger are both coaxial heat exchanger.

The types of the first microstructured reactor and the second microstructured reactor are meander reactor HC, sandwich reactor HC, fixed bed meander reactor HC or Hastelloy micro-channel reactor, respectively.

### Example 1

(1) Preparation of the Compound of the Formula III

196.2 g (2 mol) furfuryl alcohol (a compound of a formula I) was dissolved in 4 L dichloromethane, thionyl chloride (145.26 mL, 2 mol) was dropwise added into the solution at 0° C. slowly, stirring and reacting were performed at 0° C. for 1 h, and 4 L water was added to quench the reaction. An organic layer was collected and an aqueous layer was washed for three times with dichloromethane. The organic layer was combined and the solvent was spin-dried, so as to obtain colorless liquid. 184.18 g glycerol (2 mol) and 46 g sodium (2 mol) were added into the liquid and stirring and reacting were continued for 4 h at 40° C. 500 mL water was added. The organic layer was separated. The aqueous layer was extracted with toluene (250 mL*3) and the organic layer was combined. The combined organic layer was dried with anhydrous sodium sulfate and the toluene was recovered by distillation. Atmospheric distillation was carried out to obtain 292.46 g of the compound of the formula III (purity: 99.8%; yield: 85%).

(2) Preparation of the Vegetable Oil Polyol

200 g soybean oil (containing 0.99 mol of double bonds) was taken as a component I and 1360.4 g 30 wt % hydrogen peroxide (12 mol) was mixed with 563.63 g formic acid (12 mol), then 20.02 g sulfuric acid (0.2 mol, counted by H₂SO₄) and 4.38 g EDTA (0.01 mol) were added as a component II, the component I and the component II were simultaneously pumped into the first micro-mixer of the micro-channel modular reaction device at the feeding rates of 0.8 ml/min and 4.7 ml/min respectively and mixed. Then the resulted mixed solution was flowed into the first microstructured reactor and reacted. The first microstructured reactor had a volume of 44 mL and a reaction residence time of 8 min. The reaction was performed at normal pressure and 90° C., thus obtaining a reaction solution containing the epoxy vegetable oil. Next, 258 g of the compound of the formula III (1.5 mol) and the reaction solution containing the epoxy vegetable oil output by the first microstructured reactor were simultaneously pumped into the second micro-mixer of the micro-channel modular reaction device at the feeding rates of 16.6 mL/min and 5.5 mL/min respectively and mixed. Then the resulted mixed solution was flowed into the second microstructured reactor and reacted. The second microstructured reactor had a volume of 176.8 mL, a reaction residence time of 8 min and a reaction temperature of 85° C. The crude reaction product was introduced into the oil-water separator to remove the aqueous phase. Then the oil phase product was collected, thus obtaining a soybean oil polyol with the hydroxyl value of 299 mg KOH/g and the viscosity of 4736 mPa·s.

### Example 2

(1) Preparation of the Compound of the Formula III

196.2 g (2 mol) furfuryl alcohol (the compound of the formula I) was dissolved in 4 L dichloromethane, thionyl chloride (217.89 mL, 3 mol) was dropwise added into the solution at 0° C. slowly, stirring and reacting were performed at 0° C. for 2 h, and 4 L water was added to quench the reaction. An organic layer was collected and an aqueous layer was washed for three times with dichloromethane. The organic layer was combined and the solvent was spin-dried, so as to obtain colorless liquid. 184.18 g glycerol (2 mol) and 46 g sodium (2 mol) were added into the liquid and stirring and reacting were continued for 4 h at 40° C. 500 mL water was added. The organic layer was separated. The aqueous layer was extracted with toluene (250 mL*3) and the organic layer was combined. The combined organic layer was dried with anhydrous sodium sulfate and the toluene was recovered by distillation. Atmospheric distillation was carried out to obtain 309.67 g of the compound of the formula III (purity: 99.6%; yield: 90%).

(2) Preparation of the Vegetable Oil Polyol

200 g soybean oil (containing 0.99 mol of double bonds) was taken as a component I and a mixture of 1700 g 30 wt % hydrogen peroxide (15 mol) was mixed with 704.54 g formic acid (15 mol), then 30.03 g sulfuric acid (0.3 mol, counted by H₂SO₄) and 2.92 g EDTA (0.015 mol) were added as a component II, the component I and the component II were simultaneously pumped into the first micro-mixer of the micro-channel modular reaction device at the feeding rates of 0.8 ml/min and 4.7 ml/min respectively and mixed. Then the resulted mixed solution was flowed into the first microstructured reactor and reacted. The first microstructured reactor had a volume of 44 mL and a reaction residence time of 8 min. The reaction was performed at normal pressure and 90° C., thus obtaining a reaction solution containing the epoxy vegetable oil. Next, 258 g of the compound of the formula III (1.5 mol) and the reaction solution containing the epoxy vegetable oil output by the first microstructured reactor were simultaneously pumped into the second micro-mixer of the micro-channel modular reaction device at the feeding rates of 15.0 mL/min and 5.5 mL/min respectively and mixed. Then the resulted mixed solution was flowed into the second microstructured reactor and reacted. The second microstructured reactor had a volume of 164 mL, a reaction residence time of 8 min and a reaction temperature of 85° C. The crude reaction product was introduced into the oil-water separator to remove the aqueous phase. Then the oil phase product was collected, thus obtaining a soybean oil polyol with the hydroxyl value of 312 mg KOH/g and the viscosity of 4658 mPa·s.

### Example 3

(1) Preparation of the Compound of the Formula III

196.2 g (2 mol) furfuryl alcohol (the compound of the formula I) was dissolved in 4 L dichloromethane, thionyl chloride (217.89 mL, 3 mol) was dropwise added into the solution at −5° C. slowly, stirring and reacting were performed at 0° C. for 2 h, and 4 L water was added to quench the reaction. An organic layer was collected and an aqueous layer was washed for three times with dichloromethane. The organic layer was combined and the solvent was spin-dried, so as to obtain colorless liquid. 276.27 g glycerol (3 mol) and 69 g sodium (3 mol) were added into the liquid and stirring and reacting were continued for 4 h at 35° C. 500 mL water was added. The organic layer was separated. The aqueous layer was extracted with toluene (250 mL*3) and the organic layer was combined. The combined organic layer was dried with anhydrous sodium sulfate and the toluene was recovered by distillation. Atmospheric distillation was carried out to obtain 302.79 g of the compound of the formula III (purity: 99.9%; yield: 88%).

(2) Preparation of the Vegetable Oil Polyol

200 g soybean oil (containing 0.99 mol of double bonds) was taken as a component I and 1700 g 30 wt % hydrogen peroxide (15 mol) was mixed with 900.75 g acetic acid (15 mol), then 30.03 g sulfuric acid (0.3 mol, counted by H₂SO₄) and 2.92 g EDTA (0.015 mol) were added as a component II, the component I and the component II were simultaneously pumped into the first micro-mixer of the micro-channel modular reaction device at the feeding rates of 0.8 ml/min and 4.7 ml/min respectively and mixed. Then the resulted mixed solution was flowed into the first microstructured reactor and reacted. The first microstructured reactor had a volume of 44 mL and a reaction residence time of 8 min. The reaction was performed at normal pressure and 90° C., thus obtaining a reaction solution containing the epoxy vegetable oil. Next, 292 g of the compound of the formula III (1.7 mol) and the reaction solution containing the epoxy vegetable oil output by the first microstructured reactor were simultaneously pumped into the second micro-mixer of the micro-channel modular reaction device at the feeding rates of 22 mL/min and 5.5 mL/min respectively and mixed. Then the resulted mixed solution was flowed into the second microstructured reactor and reacted. The second microstructured reactor had a volume of 220 mL, a reaction residence time of 8 min and a reaction temperature of 85° C. The crude reaction product was introduced into the oil-water separator to remove the aqueous phase. Then the oil phase product was collected, thus obtaining a soybean oil polyol with the hydroxyl value of 304 mg KOH/g and the viscosity of 4895 mPa·s.

### Example 4

(1) Preparation of the Compound of the Formula III

196.2 g (2 mol) furfuryl alcohol (the compound of the formula I) was dissolved in 4 L dichloroethane, thionyl chloride (217.89 mL, 3 mol) was dropwise added into the solution at −5° C. slowly, stirring and reacting were performed at 0° C. for 2 h and 4 L water was added to quench the reaction. An organic layer was collected and an aqueous layer was washed for three times with dichloroethane. The organic layer was combined and the solvent was spin-dried, so as to obtain colorless liquid. 276.27 g glycerol (3 mol) and 69 g sodium (3 mol) were added into the liquid and stirring and reacting were continued for 4 h at 35° C. 500 mL water was added. The organic layer was separated. The aqueous layer was extracted with toluene (250 mL*3) and the organic layer was combined. The combined organic layer was dried with anhydrous sodium sulfate and the toluene was recovered by distillation. Atmospheric distillation was carried out to obtain 289.02 g of the compound of the formula III (purity: 99.5%; yield: 84%).

(2) Preparation of the Vegetable Oil Polyol

200 g grapeseed oil (containing 0.785 mol of double bonds) was taken as a component I and 1700 g 30 wt % hydrogen peroxide (15 mol) was mixed with 900.75 g acetic acid (15 mol), then 30.03 g sulfuric acid (0.3 mol, by H₂SO₄) and 2.92 g EDTA (0.015 mol) were added as a component II, the component I and the component II were simultaneously pumped into the first micro-mixer of the micro-channel modular reaction device at the feeding rates of 0.8 ml/min and 4.7 ml/min respectively and mixed. Then the resulted mixed solution was flowed into the first microstructured reactor and reacted. The first microstructured reactor had a volume of 44 mL and a reaction residence time of 8 min. The reaction was performed at normal pressure and 90° C., thus obtaining a reaction solution containing the epoxy vegetable oil. Next, 292 g of the compound of the formula III (1.7 mol) and the reaction solution containing the epoxy vegetable oil output by the first microstructured reactor were simultaneously pumped into the second micro-mixer of the micro-channel modular reaction device at the feeding rates of 19.2 mL/min and 5.5 mL/min respectively and mixed. Then the resulted mixed solution was flowed into the second microstructured reactor and reacted. The second microstructured reactor had a volume of 197.6 mL, a reaction residence time of 8 min and a reaction temperature of 85° C. The crude reaction product was introduced into the oil-water separator to remove the aqueous phase. Then the oil phase product was collected, thus obtaining a grapeseed oil polyol with the hydroxyl value of 291 mg KOH/g and the viscosity of 4959 mPa·s.

### Example 5: Performance Test of the Rigid Polyurethane Foam Prepared from the Vegetable Oil Polyol

The soybean oil polyol prepared from Example 1 was enabled to react with a foam stabilizer AK-8803 (Maysta, Nanjing), cyclohexylamine (Dajiang Chemical, Jiangdu), isocyanate WANNATE® PM-200 (Wanhua Chemical, Yantai) and a cyclopentane foaming agent (Meilong Chemical, Foshan) for foaming by a one-step free foaming process, thus preparing the rigid polyurethane foam with the apparent density of 211 kPa, the impact strength of 0.069 kJ/m² and the dimensional stability lower than 0.8%.

### Example 6

This example has the same process as Example 1, except that the mole ratio of furfuryl alcohol to thionyl chloride to glycerol to sodium is 1:1.0:1.0:1.0. Upon detection, the resulted vegetable oil polyol had similar performance with the vegetable oil polyol prepared in Example 1.

### Example 7

This example has the same process as Example 1, except that the mole ratio of furfuryl alcohol to thionyl chloride to glycerol to sodium is 1:2.0:2.0:2.0. Upon detection, the resulted vegetable oil polyol had similar performance with the vegetable oil polyol prepared in Example 1.

### Example 8

This example has the same process as Example 1, except that the catalyst was phosphoric acid, the vegetable oil was olive oil, and the mole ratio of the double bonds in the vegetable oil to hydrogen peroxide to organic acid to catalyst to stabilizer is 1:6:6:0.02:0.006. Upon detection, the resulted vegetable oil polyol had similar performance with the vegetable oil polyol prepared in Example 1.

### Example 9

This example has the same process as Example 1, except that the catalyst was phosphoric acid, the vegetable oil was peanut oil, and the mole ratio of the double bonds in the vegetable oil to hydrogen peroxide to organic acid to catalyst to stabilizer is 1:20:20:0.4:0.2. Upon detection, the resulted vegetable oil polyol had similar performance with the vegetable oil polyol prepared in Example 1.

### Example 10

This example has the same process as Example 1, except that the catalyst was phosphoric acid, and the vegetable oil was palm oil. The first microstructured reactor has a reaction temperature of 60° C., a reaction residence time of 10 min and a volume of 20 mL. The mole ratio of the epoxy groups in the epoxy vegetable oil to the compound of the formula III is 1:1.5. The second microstructured reactor has a reaction temperature of 70° C., a reaction residence time of 10 min and a volume of 96 mL. Upon detection, the resulted vegetable oil polyol had similar performance with the vegetable oil polyol prepared in Example 1.

### Example 11

This example has the same process as Example 1, except that the catalyst was phosphoric acid, and the vegetable oil was sunflower oil. The first microstructured reactor has a reaction temperature of 130° C., a reaction residence time of 5 min and a volume of 60 mL. The mole ratio of the epoxy groups in the epoxy vegetable oil to the compound of the formula III is 1:4.5. The second microstructured reactor has a reaction temperature of 100° C., a reaction residence time of 10 min and a volume of 240 mL. Upon detection, the resulted vegetable oil polyol had similar performance with the vegetable oil polyol prepared in Example 1.

What is claimed is:

1. A preparation method of a totally bio-based vegetable oil polyol, comprising enabling epoxy vegetable oil to react with a compound of a formula III in a second microstructured reactor to obtain the vegetable oil polyol

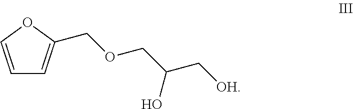 [SMILES: OCC(O)COCc1ccco1]

2. The method according to claim 1, comprising the following steps:
(1) simultaneously pumping a mixed solution of hydrogen peroxide, an organic acid, a catalyst and a stabilizer as well as the vegetable oil into a first microstructured reactor of a micro-channel modular reaction device for reacting to obtain a reaction solution containing the epoxy vegetable oil;
(2) simultaneously pumping the reaction solution containing the epoxy vegetable oil obtained from the step (1) and the compound of the formula III into the second microstructured reactor of the micro-channel modular reaction device for reacting to obtain the vegetable oil polyol

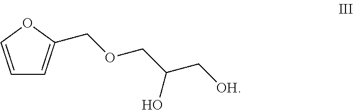 [SMILES: OCC(O)COCc1ccco1]

3. The method according to claim 2, wherein, in the step (1), the organic acid is formic acid or acetic acid, the catalyst is sulfuric acid or phosphoric acid, the stabilizer is ethylenediamine tetraacetic acid, the vegetable oil is at least one selected from olive oil, peanut oil, rapeseed oil, cottonseed oil, soybean oil, palm oil, sesame oil, sunflower oil, linseed oil, tung oil, safflower oil, rice bran oil, corn oil and teaseed oil, and the mole ratio of double bonds in the vegetable oil to the hydrogen peroxide to the organic acid to the catalyst to the stabilizer is 1:(6-20):(6-20):(0.02-0.4):(0.006-0.2).

4. The method according to claim 2, wherein, in the step (1), the first microstructured reactor has a reaction temperature of 60-130° C., a reaction residence time of 5-10 min and a volume of 20-60 mL, the vegetable oil is pumped into the micro-channel modular reaction device at a flow rate of 0.5-1.0 mL/min and the mixed solution is pumped into the micro-channel modular reaction device at a flow rate of 3.5-5.0 mL/min.

5. The method according to claim 2, wherein, in the step (2), the mole ratio of epoxy groups in the epoxy vegetable oil to the compound of the formula III is 1:(1.5-4.5), the second microstructured reactor has a reaction temperature of 70-100° C., a reaction residence time of 6-10 min and a volume of 96-240 mL, the compound of the formula III is pumped into the micro-channel modular reaction device at a flow rate of 12.0-18.0 mL/min.

6. The method according to claim 2, wherein the micro-channel modular reaction device comprises a first micro-mixer, a first microstructured heat exchanger, a first tubular temperature control module, the first microstructured reactor, a second micro-mixer, a second microstructured heat exchanger, a second tubular temperature control module and the second microstructured reactor which are sequentially connected through pipelines.

7. The method according to claim 1, wherein, in the step (2), the compound of the formula III is prepared by the following process, comprising:
(a) dissolving furfuryl alcohol in a reaction solvent, dropwise adding thionyl chloride into the solution at −10° C. to 10° C., continuing stirring and reacting for 0.5-2 h, adding water to quench the reaction, collecting an organic phase, and spin drying the reaction solvent to obtain colorless liquid;
(b) then adding glycerol and sodium into the colorless liquid, continuing stirring and reacting for 3-6 h at 30-50° C. to obtain the compound of the formula III.

8. The method according to claim 7, wherein, in the step (a), the reaction solvent is one or more of dichloromethane, dichloroethane, chloroform and benzene, and the mole ratio of furfuryl alcohol to thionyl chloride to glycerol to sodium is 1:(1.0-2.0):(1.0-2.0):(1.0-2.0).

9. A totally bio-based vegetable oil polyol wherein the totally bio-based vegetable oil polyol is prepared by a method according to claim 1.

10. A process for using a totally bio-based vegetable oil polyol of claim 9, wherein the process for using the totally bio-based vegetable oil polyol for preparing a polyurethane foam.
